SEQUENCE LISTING (14 sequences)

SEQ ID NO: 1 - DNA, 60 bp, synthetic construct
  source 1..60: mol_type = other DNA
aataaacctaaataaacctaaataaacctaaataaacctaaataaacctaaataaaccta

SEQ ID NO: 2 - DNA, 11 bp, synthetic construct
  source 1..11: mol_type = other DNA
ttcatcatcat

SEQ ID NO: 3 - DNA, 11 bp, synthetic construct
  source 1..11: mol_type = other DNA
ttcaacttacc

SEQ ID NO: 4 - DNA, 43 bp, synthetic construct
  source 1..43: mol_type = other DNA
  misc_feature 43: note = 3&apos inverted deoxythymidine
aataaacctagggccttttggccctaggtttatttaggtttat

SEQ ID NO: 5 - DNA, 43 bp, synthetic construct
  source 1..43: mol_type = other DNA
  misc_feature 43: note = 3&apos inverted deoxythymidine
acatcatcatgggccttttggcccatgatgatgtatgatgatg

SEQ ID NO: 6 - DNA, 43 bp, synthetic construct
  source 1..43: mol_type = other DNA
  misc_feature 43: note = 3&apos inverted deoxythymidine
acaacttaacgggccttttggcccgttaagttgtgttaagttg

SEQ ID NO: 7 - DNA, 20 bp, synthetic construct
  source 1..20: mol_type = other DNA
cgaaacaaagtacaacggag

SEQ ID NO: 8 - DNA, 20 bp, synthetic construct
  source 1..20: mol_type = other DNA
gcctgattgctttgaatacc

SEQ ID NO: 9 - DNA, 20 bp, synthetic construct
  source 1..20: mol_type = other DNA
gaaataccgaccgtgtgata

SEQ ID NO: 10 - DNA, 20 bp, synthetic construct
  source 1..20: mol_type = other DNA
taagaggtcatttttgcgga

SEQ ID NO: 11 - DNA, 20 bp, synthetic construct
  source 1..20: mol_type = other DNA
gccatatttaacaacgccaa

SEQ ID NO: 12 - DNA, 20 bp, synthetic construct
  source 1..20: mol_type = other DNA
tctgtccatcacgcaaatta

SEQ ID NO: 13 - DNA, 20 bp, synthetic construct
  source 1..20: mol_type = other DNA
cggaacaacattattacagg

SEQ ID NO: 14 - DNA, 20 bp, synthetic construct
  source 1..20: mol_type = other DNA
gagagataacccacaagaat

(19) United States
(12) Patent Application Publication
(10) Pub. No.: US 2025/0109392 A1
(43) Pub. Date: Apr. 3, 2025

(54) GENERAL CHEMICAL SYSTEM FOR UNIVERSALLY AUGMENTING THE FUNCTION OF PROTEINS

(71) Applicant: The Chinese University of Hong Kong, Hong Kong (CN)

(72) Inventors: Hei Ming LAI, Hong Kong (CN); Pui Lam TSOI, Tsuen Wan (CN); Chun Ngo YAU, Hong Kong (CN)

(21) Appl. No.: 18/901,841

(22) Filed: Sep. 30, 2024

Related U.S. Application Data
(60) Provisional application No. 63/586,483, filed on Sep. 29, 2023.

(51) Int. Cl. C12N 9/96

(52) U.S. Cl. CPC C12N 9/96

(57) ABSTRACT

A supramolecular complex augmentor, comprising at least one guest molecule and at least one guest molecule that forms a weak guest-host complex. The complex can be of a solubilizing bile salt detergent weakly complexed to at least one cyclodextrin derivative, where addition of the augmentor to an environment comprising at least one protein enhances the in vitro performance of the protein.

### CROSS-REFERENCE TO RELATED APPLICATION

This application claims the benefit of U.S. Provisional Application Ser. No. 63/586,483, filed Sep. 29, 2023, which is hereby incorporated by reference in its entirety including any tables, figures, or drawings.

### SEQUENCE LISTING

The Sequence Listing for this application is labeled “CUHK-224X-SeqList.xml” which was created on Sep. 18, 2024, and is 14,500 bytes. The entire contents of the sequence listing is incorporated herein by reference in its entirety.

### BACKGROUND OF THE INVENTION

Proteins are ubiquitous in the biotechnology industry. The maturation of molecular cloning, protein expression, directed evolution, and protein design technologies have promoted proteins as a universal reagent of choice in diverse domestic, industrial, and scientific applications. Although significant progress has been made in the generation or even de novo design of proteins with desired functions, there has been little effort to enhance their functions without altering their peptide sequences. To this end a method of augmenting native protein function by inclusion of an additive without altering the peptide sequence is desirable.

### BRIEF SUMMARY OF THE INVENTION

A supramolecular system is employed to augment native protein functions that can boost their baseline activities by 2-5 times when the supramolecular agent is added to the protein at its optimally performing conditions. The system's acronym is AUGMENT (for augmentative Supramolecular system for general activity multiplicative enhancement), the system involves an addition of sub-millimolar concentrations of a zwitterionic bile salt derivative and cyclodextrin, and is applicable in diverse contexts, from in vitro enzyme assays to tissue-based ex vivo applications such as immunostaining.

### BRIEF DESCRIPTION OF THE DRAWINGS

FIG. 1A shows a chemical structure of the CHAPSO detergent.

FIG. 1B shows a chemical structure of 6-peraminated-β-cyclodextrin (6NβCD)

FIG. 1C shows a composite of electrostatic potential maps of CHAPSO and 6NβCD.

FIG. 1D shows a schematic of the association-dissociation of the CHAPSO-6NβCD supramolecular complex.

FIG. 1E is a plot of the augmentation of diverse enzymes' native activities by the CHAPSO-6NβCD supramolecular complex.

FIG. 2 shows plots indicating no obvious correlation on the enzymes' characteristics with their augmentation.

FIG. 3A shows a Venn diagram detailing the number of commercially available antibodies that worked with: control staining (PBST); only with AUGMENT; or neither.

FIG. 3B shows immunofluorescence images where antibody specific staining patterns of DAPI and GFAP were recovered by in situ application of the AUGMENT system in mouse tissue.

FIG. 3C shows examples of immunofluorescence images CD19 and CD68 where antibody specific staining patterns were recovered by in situ application of the AUGMENT system in human tissue.

FIG. 3D shows quantified recovered fluorescent protein signal from in situ-denatured tissue using AUGMENT with EYFP, dsRed, and EGFP.

FIG. 3E shows quantified recovered immunoreactivity signal from in situ-denatured tissue using AUGMENT, with anti-PV, anti-VIP, anti-pS6, and anti-ChAT.

### DETAILED DISCLOSURE OF THE INVENTION

A protein AUGMENT system is derived from a bile salt-cyclodextrin artificial chaperone system. The system includes at least one weak host-guest complex. The molecular host can be, but is not limited to, at least one cucurbiturils, cyclodextrins, calixarenes, cyclophanes, cryptands, and cryptophanes or derivative thereof. The molecular guest has an activity that can be one or more molecules that inhibit aggregation of denatured antibodies as well as inhibit the interactions between antigen and functional antibodies. The molecular guest can be a protein solubilizing bile salt, a detergent, strongly complexes with cyclodextrins to form an artificial chaperone system. In specific embodiments, an ionic or zwitterionic bile salt and a charged cyclodextrin derivative with weak complexation, having an association constant <10³ M⁻¹, comprise an augmentor when added in one step to provide continuous refolding of proteins in their native form and environment. In this manner the protein's activity is augmented by selectively favoring the equilibrium towards the soluble pool of proteins, rather than being recovered to its “native” protein activity. This result is equivalent to repeatedly and continuously applying the bile salt-cyclodextrin artificial chaperone system to a protein solution. Both components of the augmentor are added together into the proteins' in vitro reaction environment to obtain a gain in the proteins' performance. In some cases, the proteins' performance can be enhanced outside of their usually specified condition (e.g., suggested optimal reaction by the vendors or established methods, such as reaction pH, ionic strength, osmolarity, reaction temperature, substrate concentration, inhibitor concentration, denaturant concentration, the use of other additives and their concentrations etc.).

The concentration of the molecular host(s) and molecular guest(s) of the augmentor in a reaction medium of one or more proteins can vary depending on the enhanced performance desired, which can be determined by a standardized assay method that establishes a range of optimal concentrations for enhancing the protein's function. In the case of a protein mixture system (where multiple proteins are necessarily present, e.g., in the case of a cell-free in vitro protein synthesis system), an overall optimal concentration of the host and guest molecules of the augmentor(s) can be similarly determined and applied.

This augmentor system is the first demonstration that simultaneous addition of both a guest detergent and its complexing supramolecular host can lead to direct enhancement or augmentation of protein activity in its native operating environment, and is continuously present as an additive throughout the proteins reaction assay or performing environment. This contrasts to other artificial chaperones which requires two-step recovery of the protein's function from a denatured/non-functional state, and requires purification of the protein to remove artificial chaperone components before using the protein in its respective applications.

The augmentor benefits all of the six enzyme classes of the Enzyme Commission, and with antibody-based applications such as immunohistochemistry, ELISA, and immunoprecipitation, multi-protein system such as multistep enzymatic reactions, and in vitro protein synthesis/cell-free protein synthesis system.

In embodiments, the derivatives of bile salts include, but are not limited to, derivatives of cholic acid (3β, 7β, 12β-trihydroxycholanoic acid), itself, its C3, C7, C12 epimers and combinations thereof, its C3, C7, and C12 deoxy derivatives and combinations of the epimers thereof, their taurine or glycine conjugates, and derivatives 3-([3-cholamidopropyl]dimethylammonio)-2-hydroxy-1-propanesulfonate and 3-([3-cholamidopropyl]dimethylammonio)-1-propanesulfonate, commonly known as CHAPSO and CHAPS, respectively. General structures are displayed in Structure 1 below.

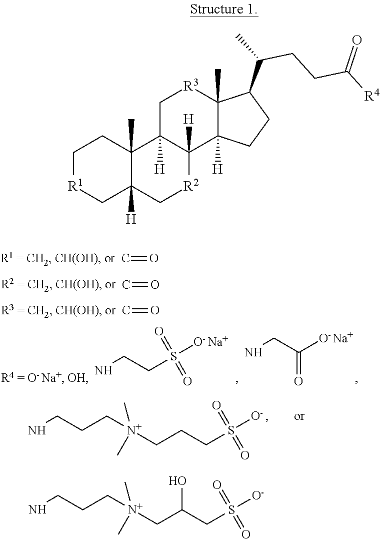

In embodiments, the derivatives of cyclodextrins include, but are not limited to, hexakis-β-glucopyranose (also known as α-cyclodextrin), heptakis-β-glucopyranose (also known as β-cyclodextrin), octakis-β-glucopyranose (also known as γ-cyclodextrin), and their derivatives. The derivatives of hexakis-β-glucopyranose, include mono-, bis-, tris-, tetrakis-, pentakis-, and hexakis-substitution, and additionally, heptakis- and octakis-substitution for heptakis-β-glucopyranose and octakis-β-glucopyranose, respectively, of the hydroxyl groups at C2, C3, C6 positions, in isolation or in combination. The derivatives need not be used as a single, pure isomer but can be a mixture of isomers. Substitution of cyclodextrin hydroxy-groups can be amino-, methoxy-. (2-hydroxy)propoxyl, sulphato-, guanidino-, acetato-, acetamido-, azido-, bromo-, iodo-, chloro-, toluenesulfonyl-, thio-, succinyl-, phosphato-, 4-sulfatobutoxy-, carboxymethoxy- and (2-aminoethyl)amino-groups.

the general structure is displayed in structure 2 below.

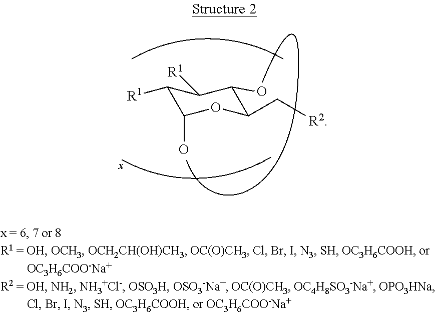

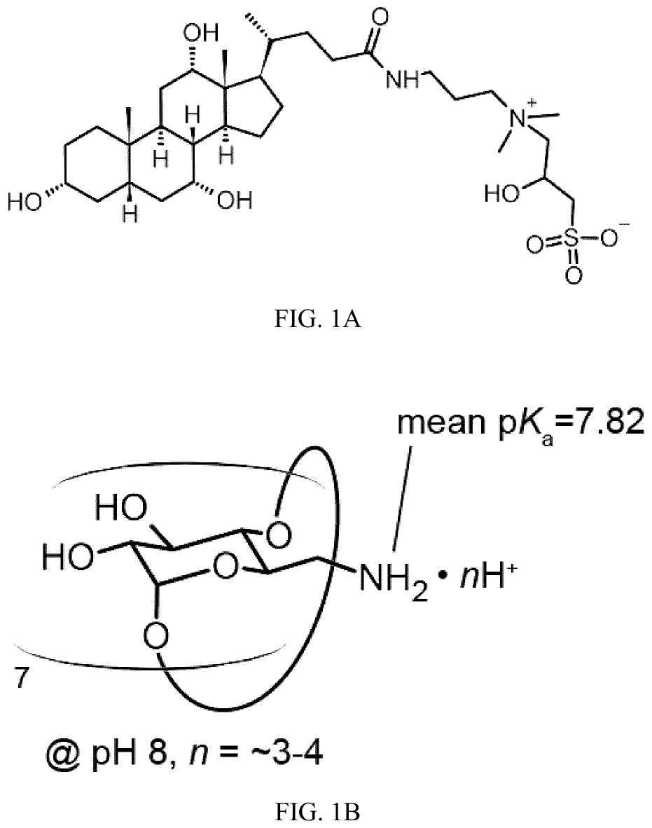

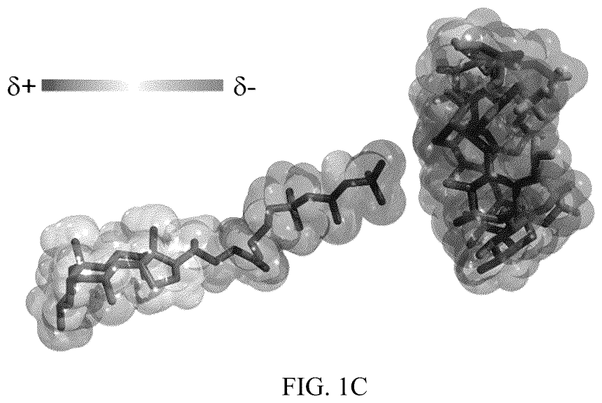

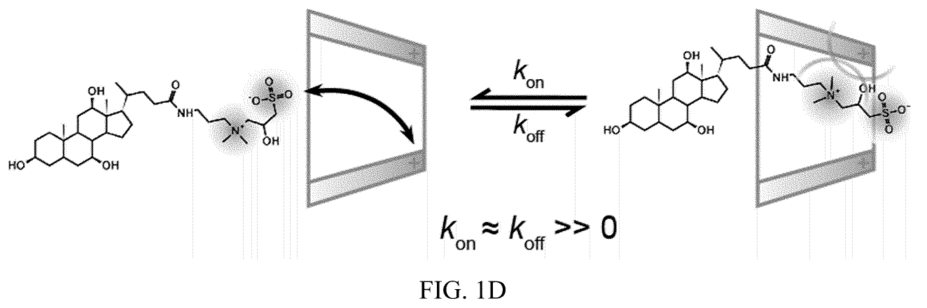

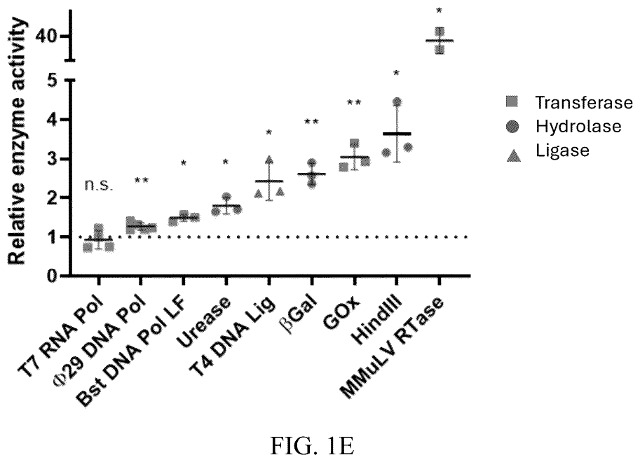

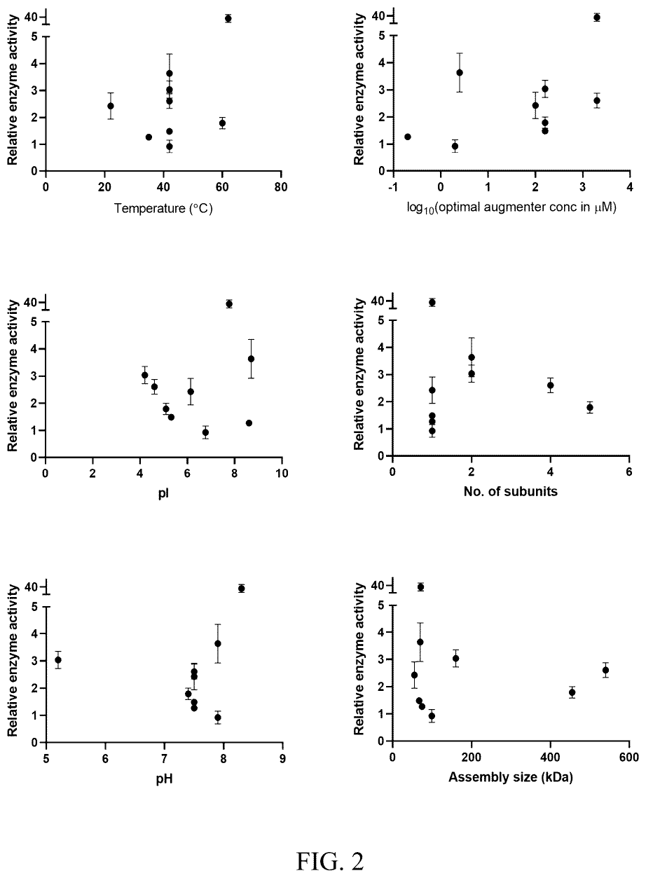

A prior art deoxycholate/β-cyclodextrin system demonstrated a rapidly dissociating-associating process at 55° C. According to an embodiment, a similar rapid on-off binding phenomena at ambient temperatures results from including a zwitterionic detergent, CHAPSO (3-([3-cholamidopropyl]dimethylammonio)-2-hydroxy-1-propanesulfonate), shown in FIG. 1A, and heptakis-(6-amino-6-deoxy)-β-cyclodextrin (6NβCD), shown in FIG. 1B. Alternatively, heptakis-(6-amino-6-deoxy)-γ-cyclodextrin (6NγCD) allows similar results with CHAPSO as an augmentor. This mixture, an augmentor, forms a highly dynamic supramolecular complex with a low complex formation constant. Nuclear Magnetic Resonance (NMR) spectroscopy and ROESY (rotating-frame nuclear Overhauser effect correlation spectroscopy) analysis suggest the existence of weak reversible complex of less than 10³ M⁻¹, both at room temperature (RT) and at 45° C., as shown in FIGS. 1C and 1D, that in stark contrast to the prior art deoxycholate-βCD system, which has slow off-kinetics at RT.

Various concentrations of CHAPSO-6NβCD added to various enzyme reaction assays, display a peak increased performance that typically exceeds the native condition as indicated in FIG. 1E. In FIG. 1E augmentation is shown to vary from showed no augmentation, although no inhibition for T7 RNA polymerase to augmentation for M-MuLV reverse transcriptase that shows a 40-fold activity augmentation, while other enzymes show augmentation that ranged from 1.37 to 3.64 fold. The augmentation can be carried out for any enzyme class, isoelectric point, concentration of augmentor used, the temperature and pH of operation as indicated in the plot for temperature, isoelectric point (pI), augmentor concentration, number of subunits, pH, and assembly size for augmentation for Phi29 DNA polymerase in FIG. 2. Although the augmentation effects is less pronounced with some enzymes with the CHAPSO-6NγCD system the augmentation is statistically significant in most cases.

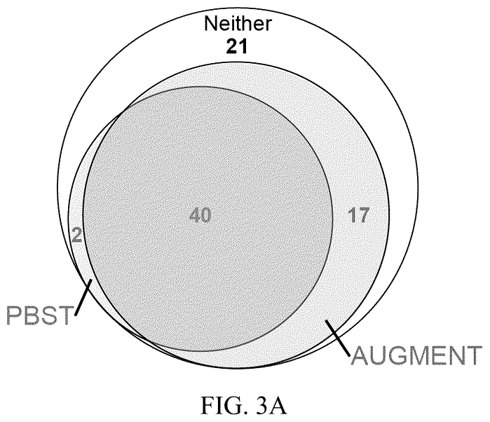

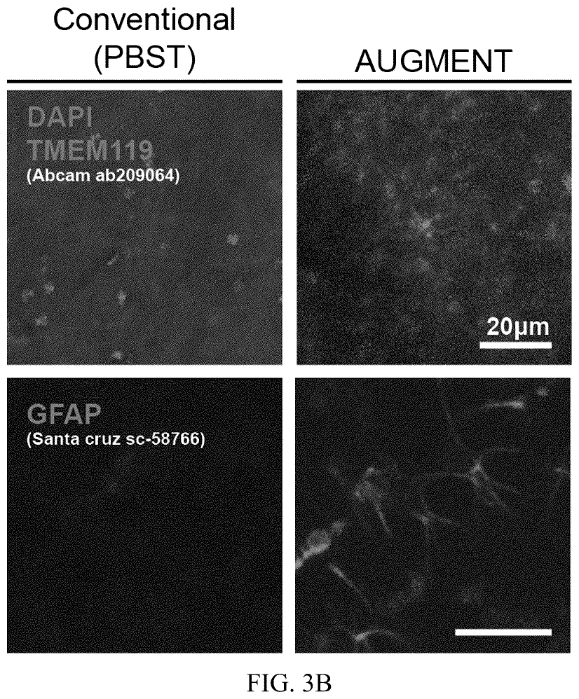

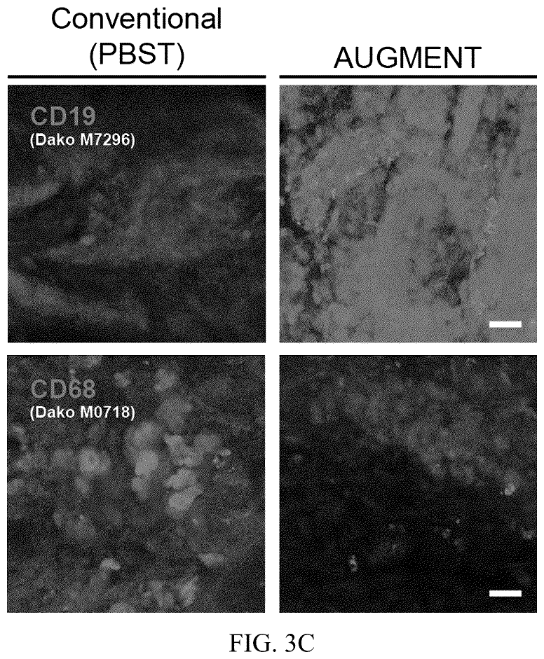

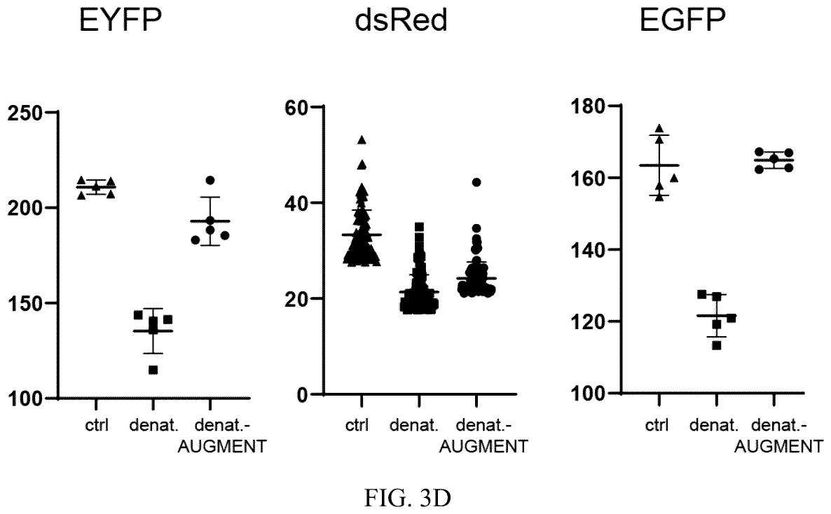

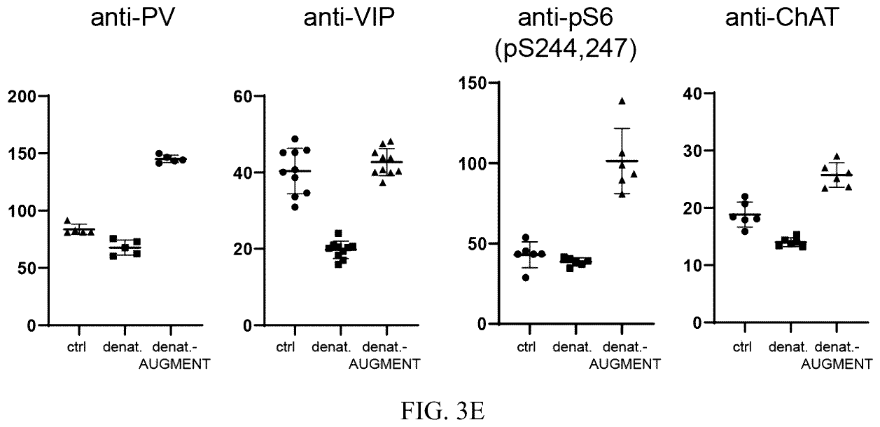

The CHAPSO-6NβCD and the CHAPSO-6NγCD systems are applicable to augment antibody performance in in situ settings, such as immunofluorescence, when applied at a final concentration of 6 mM in Tris-CAPS buffer compared to a control staining buffer (PBS with 0.1% w/v Triton X-100), as shown in FIGS. 3A through 3C. For example, as indicated in Table 1, below, of 80 antibodies on mouse and human tissues, 17 antibodies produce specific immunofluorescence signals when the corresponding control staining buffer did not produce signals as shown in FIG. 3A through 3C. The CHAPSO-6NβCD system allows recovery of denatured tissue antigens in situ. Tissues treated in 6M guanidinium chloride at 60° C. for 1 hour before proceeding to immunostaining in Tris-CAPS buffer with or without CHAPSO-6NβCD show that the presence of CHAPSO-6NβCD not only recovers but display improved immunofluorescence signals as shown in FIG. 3D, and rescue endogenous fluorescent protein signals when they are present as shown in FIG. 3E. A recovery of immunofluorescence performance does not occur with other cyclodextrin derivatives and is lower using other cholic acid derivatives.

TABLE 1
Performance of antibodies in producing specific immunofluorescence (IF) patterns
under control IF or augmented IF conditions.

| Antibody | Company (cat. no.) | Tissue used | Control IF | Augmented IF |
|---|---|---|---|---|
| Rb anti-Asyn | Abcam (ab138501) | Human Brain | Yes | Yes |
| Rb anti-CD31 | Abcam (ab28364) | Human Brain | No | No |
| Gt anti-PV | Abcam (ab32895) | Human Brain | No | No |
| Rt anti-SST | Millipore (MAB354) | Human Brain | No | Yes |
| Rb anti-DLG3 | Sigma (HPA001733) | Human Brain | No | Yes |
| Rb anti-DBH | Sigma (HPA002130) | Human Brain | Yes | Yes |
| Rb anti-OLIG2 | Sigma (HPA003254) | Human Brain | No | Yes |
| Rb anti-GABARA1 | Sigma (HPA055746) | Human Brain | No | Yes |
| Rb anti-VIP | Bioss (bs-0077R) | Human Brain | Yes | Yes |
| Ms anti-p40ANp63 | Abcam (ab172731) | Human Tumor | Yes | Yes |
| Rb anti-CD4 | AbClonal (A19018) | Human Tumor | Yes | Yes |
| Rb anti-CD44 | AbClonal (A19020) | Human Tumor | Yes | Yes |
| Rb anti-CD79a | AbClonal (A19024) | Human Tumor | No | Yes |
| Ms anti-CD68 | Dako (M0718) | Human Tumor | No | Yes |
| Rb anti-CD3 | Dako (M7254) | Human Tumor | No | Yes |
| Ms anti-CD19 | Dako (M7296) | Human Tumor | No | Yes |
| Ms anti-CD56 | Dako (M7304) | Human Tumor | No | Yes |
| Rb anti-Cytokeratin 8 | AbClonal (A1024) | Human Tumor | Yes | Yes |
| Rt anti-CD8a | Invitrogen (14-0808-82) | Human Tumor | Yes | Yes |
| Rb anti-NPHS2 | Abcam (ab50339) | Mouse Kidney | No | No |
| Gt anti-S100A10 | R&D (AF2377) | Mouse Kidney | No | No |
| Rb anti-LRRK2 | Abcam (ab133474) | Mouse Kidney | No | No |
| Rb anti-NaK-ATPase | AbClonal (A11683) | Mouse Kidney | Yes | Yes |
| Rb anti-TOM20 | AbClonal (A19403) | Mouse Kidney | Yes | Yes |
| Rb anti-Vimentin | AbClonal (A19607) | Mouse Kidney | Yes | Yes |
| Rb anti-TACC3 | AbClonal (A19617) | Mouse Kidney | No | Yes |
| Rb anti-Calbindin | CST (13176S) | Mouse Kidney | No | No |
| Ms anti-aSMA | Invitrogen (MA1-06110) | Mouse Kidney | Yes | Yes |
| Ms anti-CD44 | LSBio (LS-C359273) | Mouse Kidney | Yes | Yes |
| Rb anti-Podocin | Sigma (P0372) | Mouse Kidney | No | No |
| Gt anti-Jagged1 | R&D (AF599) | Mouse Kidney | No | No |
| Ms anti-PINK1 | Abcam (ab186303) | Mouse Kidney | Yes | Yes |
| Rb anti-CD34 | AbClonal (A0761) | Mouse Kidney | Yes | Yes |
| Rb anti-TMEM119 | Abcam (ab209064) | Mouse Brain | No | Yes |
| Rb anti-Arc | Synaptic systems (156 003) | Mouse Brain | Yes | Yes |
| Ms anti-NeuN | Abcam (ab104224) | Mouse Brain | Yes | Yes |
| Rb anti-PARK7 | Abcam (ab18257) | Mouse Brain | No | No |
| Rb anti-OCT4 | Abcam (ab19857) | Mouse Brain | Yes | Yes |
| Rb anti-aSMA | Progen (690001) | Mouse Brain | No | Yes |
| Ms anti-ALDH1L1 | Abcam (ab56777) | Mouse Brain | No | Yes |
| Sh anti-aSyn | Abcam (ab6162) | Mouse Brain | Yes | Yes |
| Ms anti-bIIITub | Abcam (ab78078) | Mouse Brain | Yes | Yes |
| Rb anti-VIP | Abcam (ab8556) | Mouse Brain | Yes | Yes |
| Rb anti-SOX2 | Abcam (ab97959) | Mouse Brain | No | No |
| Rb anti-SOD2 | AbClonal (A19576) | Mouse Brain | Yes | Yes |
| Ms anti-GFAP | CST (3670S) | Mouse Brain | No | Yes |
| Rb anti-Phospho-p44/42 MAPK | CST (4370S) | Mouse Brain | No | No |
| Rb anti-S100b | Enzo (ENZ-ABS307) | Mouse Brain | Yes | No |
| Rb anti-DBH | Immunostar (22806) | Mouse Brain | Yes | Yes |
| Ms anti-TH | Immunostar (22941) | Mouse Brain | No | No |
| Rt anti-GFAP | Invitrogen (13-0300) | Mouse Brain | Yes | Yes |
| Rb anti-Phospho-S6 (S244, S247) | Invitrogen (44-923G) | Mouse Brain | Yes | Yes |
| Rb anti-PV | Invitrogen (PA1-933) | Mouse Brain | Yes | Yes |
| Rb anti-PTX3 | Invitrogen (PA5-101097) | Mouse Brain | No | No |
| Rb anti-SLC5A3 | Invitrogen (PA5-101896) | Mouse Brain | Yes | Yes |
| Rb anti-NPAS4 | Invitrogen (PA5-39300) | Mouse Brain | Yes | Yes |
| Ms anti-SLC1A2 | LSBio (LS-B6724) | Mouse Brain | Yes | Yes |
| Rb anti-SIPR3 | LSBio (LS-C406078) | Mouse Brain | Yes | Yes |
| Ms anti-NeuN | Milipore (MAB377) | Mouse Brain | No | Yes |
| Gt anti-ChAT | Millipore (AB144) | Mouse Brain | Yes | Yes |
| Rb anti-TH | Millipore (AB152) | Mouse Brain | Yes | Yes |
| Ms anti-PSD95 | NeuroMab (75-028) | Mouse Brain | No | No |
| Rb anti-Synapsin I | Novus (NB300-104) | Mouse Brain | Yes | Yes |
| Gt anti-Calretinin | R&D (AF5065) | Mouse Brain | No | No |
| Ms anti-TH | R&D (MAB7566) | Mouse Brain | No | No |
| Ms anti-c-Fos | SantaCruz (sc-166940) | Mouse Brain | Yes | Yes |
| Ms anti-Gephyrin | SantaCruz (sc-25311) | Mouse Brain | No | No |
| Rt anti-SST | Milipore (MAB354) | Mouse Brain | Yes | Yes |
| Ms anti-GFAP | SantaCruz (sc-58766) | Mouse Brain | No | Yes |
| Rb anti-GABA | Sigma (A2052) | Mouse Brain | Yes | Yes |
| Ms anti-VGLUT2 | Sigma (AMAB91081) | Mouse Brain | No | No |
| Ms anti-TPH2 | Sigma (AMab91108) | Mouse Brain | Yes | Yes |
| Ms anti-Calbindin D28K | Sigma (C9848) | Mouse Brain | No | Yes |
| Rb anti-ALDOC | Sigma (HPA003282) | Mouse Brain | Yes | Yes |
| Rb anti-DDC | Sigma (HPA017742) | Mouse Brain | No | No |
| Ms anti-Calbindin D28K | Swant (CB300) | Mouse Brain | Yes | Yes |
| Rb anti-EAAT2 | Abcam (ab41621) | Mouse Brain | Yes | Yes |
| Ms anti-MAP2 | Abcam (ab11267) | Mouse Brain | Yes | No |
| Gt anti-Iba1 | Abcam (ab5076) | Mouse Brain | No | No |
| Rt anti-C3 | Abcam (ab11862) | Mouse Brain | No | No |

To investigate the mechanisms of CHAPSO-6NβCD system in protein augmentation, a complementary augmentation system with the electrostatic charges are flipped. This system includes a novel cholic acid derivative 3-cholamidoethoxy(trimethylammoniumethanol)phosphate (CHAMP) and the 6-per-sulphated βCD derivative, heptakis(6-sulfo)-β-cyclodextrin sodium salt (hereafter abbreviated as 6SβCD). NMR and ROESY analysis show that CHAMP is weakly complexed by 6SβCD, presumably due to the colliding electrostatic charges. When applied to a glucose oxidase and urease assay, the CHAMP-6SβCD also resulted in augmentation of enzyme activity by a similar degree and at similar concentrations.

### Materials and Methods

CHAPSO was purchased and used as received from Santa Cruz Biotechnology (sc-280635B). Cyclodextrins and their derivatives were purchased either from Cyclolab or Arachem Cyclodextrin Shop and used as received.

All NMR spectra were recorded on Bruker spectrometer with operating frequencies of 150(¹³C) and 600 (¹H) MHz. The spectra are referred to the residual solvent signal (D₂O, d4-MeOD). The data for ¹H NMR is represented as follows: chemical shift (δ, ppm), multiplicity (s=singlet, d=doublet, t=triplet, m=multiplet, br=broad singlet, coupling constant (s) in Hz, integration). Data for ¹³C{¹H} are expressed in terms of chemical shift (δ, ppm). High-resolution mass spectra were recorded using ESI or APCI ionization on a Q Exactive Focus Orbitrap spectrometer from Thermo Scientific.

### Synthesis of CHAMP

CHAMP (2-((R)-4-((3R,5S,7R,8R,9S,10S,12S,13R,14S,17R)-3,7,12-trihydroxy-10,13- dimethylhexadecahydro-1H-cyclopenta[a]phenanthren-17-yl)pentanamido)ethyl-(2-(trimethylammonio)ethyl)phosphate) was synthesized according to the following scheme:

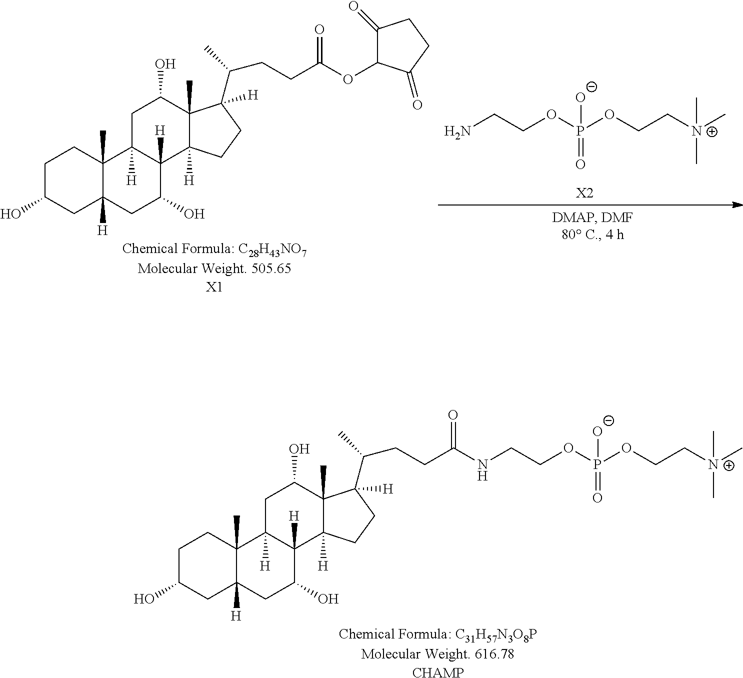

Where X1 was synthesized from cholic acid following the procedure in M. Zhang, K. C. Waldron, X. X. Zhu, RSC Advances 2016, 6, 35436-35440.

X2 (281 mg, 1.25 mmol, 1.05 eq., from Sage Chem Ltd, CAS 82755-93-9) was dried by) co-evaporation with acetonitrile (2 mL) and placed under a high vacuum for 2 h. Dry DMF (1.5 mL) was added, followed by addition of activated ester X1 (600 mg, 1.18 mmol, 1.00 eq.) and DMAP (14.5 mg, 0.12 mmol, 10 mol %). The reaction was stirred for 4 h at 80° C. The reaction mixture was then directly subjected to reversed column chromatography (C-18 SiO₂, MeOH) yielding CHAMP as a white solid (296 mg, 40%).

R_{f}=0.05 (DCM/MeOH, 3:1); ¹H-NMR (600 MHZ, MeOH-d4): δ [ppm]=4.25-4.29 (m, 2H), 3.94-3.97 (m, 1H), 3.92 (dd, J=6.0, 12.7 Hz, 2H), 3.78-3.80 (m, 1H), 3.62-3.66 (m, 2H), 3.36-3.43 (m, 3H), 3.23 (s, 9H), 2.22-2.33 (m, 3H), 2.09-2.17 (m, 1H), 1.71-2.05 (s, 7H), 1.63-1.68 (m, 1H), 1.50-1.62 (m, 5H), 1.26-1.47 (m, 5H), 1.08-1.16 (m, 1H), 1.03 (d, J=6.9 Hz, 3H), 0.98 (dt, J=3.4, 14.0 Hz, 1H), 0.92 (s, 3H), 0.72 (s, 3H); ¹³C{¹H}-NMR (150 MHz, MeOH-d4): δ [ppm]−177.0, 74.0, 72.9, 69.0, 67.5, 65.2 (d, J=4.8 Hz), 60.4 (d, J=5.1 Hz), 54.7, 48.0, 47.5, 43.2, 43.0, 41.3, 41.2, 41.0, 40.5, 37.0, 36.5, 35.9, 34.1, 33.3, 31.2, 29.6, 28.7, 27.9, 24.2, 23.2, 17.8, 13.0; HRMS (ESI) m/z: [M+H]⁺ Calcd for C₃₁H₅₇N₂O₈P H⁺ 617.39253, found 617.39143; [M+Na]⁺ Calcd for C₃₁H₅₇N₂O₈P Na⁺ 639.37447, found 639.37316.

### Enzymatic Activity Assays

HindIII restriction endonuclease activity was measured using a partial digest of Lambda phage genomic DNA (TakaraBio, cat no. 3010) as the substrate. Reaction mixtures (5 μl) contained 1× NEB 2.1 buffer (New England Biolabs, cat no. B7202S), 0.25 μg DNA substrate, CHAPSO and 6NβCD at the indicated concentrations, and 0.1 U HindIII (New England Biolabs, cat no. R3104S). Reactions were incubated at 42° C. for 1 h, then halted by heating to 65° C. for 10 min. Reaction products (5 μl) were resolved on a 0.5% agarose gel in TBE buffer run at 4 V/cm for 4 h, stained with GelRed (Biotium), and imaged using a BioRad Gel Doc EZ imager.

β-galactosidase activity was determined using a colorimetric method. Reaction mixtures (6 μl) contained 1× phosphate buffer (0.6 M Na₂HPO₄, 0.4 M NaH₂PO₄, 0.1 M KCl, 0.01 M MgSO₄, pH 7.5), 1 mg/mL X-gal, CHAPSO and 6NβCD at the indicated concentrations, and 10 mU β-galactosidase (Sigma-Aldrich, cat no. G5635). Reactions were incubated at 42° C. for 1 h, then halted by adding an equal volume of 20% Triton X-100 to dissolve the precipitated chromophore. The absorbance spectra from 525 to 775 nm were measured using a NanoDrop 2000 spectrophotometer, and the area under the curve (AUC) was calculated after subtracting baselines determined from 500 to 524 nm and 776 to 800 nm absorbance values.

Jackbean Urease activity was measured using a modified Berthelot colorimetric assay. Reaction mixtures (8 μl) contained 20 mM sodium phosphate buffer (pH 7.4), 60 mM sodium salicylate, 3.4 mM sodium nitroprusside, 0.1 mg/mL urea, CHAPSO and 6NβCD at the indicated concentrations, and 0.5 mU urease (Sigma-Aldrich, cat no. U1500). Reactions were incubated at 60° C. for 30 min, then halted by adding an equal volume of alkaline hypochlorite solution (10 mM sodium hypochlorite, 150 mM NaOH). Absorbance spectra from 550 to 840 nm were measured and AUC was calculated as for the β-galactosidase assay.

Bst DNA polymerase large fragment (LF) activity was measured using a primer exchange reaction (PER) assay. Reaction mixtures (10 μl) contained 1×PBS, 10 mM MgSO₄, 0.3 mM each dATP, dCTP and dTTP, 0.5 μM 5′-biotinylated hairpin oligo, 5 μM probe oligo, CHAPSO and 6NβCD at the indicated concentrations, and 3 U Bst DNA pol LF (MCLAB, cat no. BPL-300). Reactions were incubated at 42° C. for 2 h, then halted by heating to 80° C. for 20 min. Reaction products (6 μl) were resolved on a 1.5% agarose gel and imaged as described above. PER was performed in triplicate using different sets of hairpin and probe oligonucleotides with sequences listed in Table 1, below.

T7 RNA polymerase activity was measured using an in vitro transcription assay with a plasmid template encoding the T7 promoter. Reaction mixtures (10 μl) contained 0.25 μg template plasmid, 1× T7 reaction buffer (MCLAB), 0.5 mM NTP mix (Invitrogen, cat no. 18109-017), 10 mM dithiothreitol, 2 mM spermidine, CHAPSO and 6NβCD at the indicated concentrations, and 20 U T7 RNA polymerase (MCLAB, cat no. RP-200). Reactions were incubated at 42° C. for 2 h, then 2 μl were transferred to 98 μl nuclease-free water in a black 96-well plate. RNA concentration was measured using the Quant-iT RiboGreen RNA Assay (Thermo Fisher Scientific, cat no. R11490) according to the manufacturer's instructions. Fluorescence (485 nm excitation, 535 nm emission) was measured using a Wallac 1420 plate reader.

Phi29 DNA polymerase activity was measured using a rolling circle amplification assay. Reaction mixtures (10 μl) contained 20 ng M13mp18 ssDNA, 0.5 μM each of 8 antisense primers to M13mp18 sequences of Table 1, below, 1× Phi29 polymerase reaction buffer (MCLAB), 1 mM each dNTP, CHAPSO and 6NβCD at the indicated concentrations, and 10 U Phi29 DNA polymerase (MCLAB, cat no. PP-200). Reactions were incubated at 35° C. for 16 h, then halted by heating to 65° C. for 10 min. Reaction products (5 μl) were resolved on a 1% agarose gel and imaged as described above.

TABLE 1
Sequences of oligos used.
PER primer oligos

| | |
|---|---|
| 5Am-26x6 | /5AmMC6/[AATAAACCTA]₆ (SEQ ID NO: 1) |
| p27 | TTCATCATCAT (SEQ ID NO: 2) |
| p28 | TTCAACTTACC (SEQ ID NO: 3) |

PER hairpin oligos

| | |
|---|---|
| h.26.26 | AATAAACCTAGGGCCTTTTGGCCCTAGGTTTATTTAGGTTT |
| | AT/3InvdT/ (SEQ ID NO: 4) |
| h.27.27 | ACATCATCATGGGCCTTTTGGCCCATGATGATGTATGATGA |
| | TG/3InvdT/ (SEQ ID NO: 5) |
| h.28.28 | ACAACTTAACGGGCCTTTTGGCCCGTTAAGTTGTGTTAAGT |
| | TG/3InvdT/ (SEQ ID NO: 6) |

M13mp18 antisense primers

| | |
|---|---|
| M13-1 | CGAAACAAAGTACAACGGAG (SEQ ID NO: 7) |
| M13-2 | GCCTGATTGCTTTGAATACC (SEQ ID NO: 8) |
| M13-4 | GAAATACCGACCGTGTGATA (SEQ ID NO: 9) |
| M13-5 | TAAGAGGTCATTTTTGCGGA (SEQ ID NO: 10) |
| M13-6 | GCCATATTTAACAACGCCAA (SEQ ID NO: 11) |
| M13-7 | TCTGTCCATCACGCAAATTA (SEQ ID NO: 12) |
| M13-8 | CGGAACAACATTATTACAGG (SEQ ID NO: 13) |
| M13-9 | GAGAGATAACCCACAAGAAT (SEQ ID NO: 14) |

MMuLV reverse transcriptase (RTase) assay was performed using two methods: a fluorescent assay to measure cDNA synthesis and ethanol precipitation to quantify total nucleic acids. For the fluorescent assay, the reaction mixture contained 20 ng mouse reference RNA (Thermo Fisher, cat no. QSO640), 1 mM dNTPs (Takara Bio), 1 μM oligo dT₃₀ primer (Integrated DNA Technologies), 1× first-strand buffer (MCLAB), 1 mM DTT (Takara Bio, cat no. ST0063), and MMuLV RTase (MCLAB, cat no. SMRT-300) in a total volume of 10 μl. The RNA, dNTPs, and primer were first annealed by heating to 65° C. for 5 minutes. CHAPSO and 6NβCD were added to the indicated conditions, followed by 0.5 mU of RTase. Reactions were incubated at 52° C. for 1.5 hours, then at 85° C. for 5 minutes to inactivate the RTase. Two and a half microliters of each reaction was added to 97.5 μl of water in a black 96-well plate, then 100 μl of OliGreen working solution (Thermo Fisher, cat no. O7582) was added. Fluorescence was measured using a Wallac 1420 fluorescence plate reader with excitation at 485 nm and emission at 535 nm. For the ethanol precipitation assay, the reactions were scaled up to 25 μl and contained the same reagents at the same concentrations. After the incubation and inactivation steps, 4 μg of RNase A (Sigma, cat no. R5125) was added and reactions were incubated at 37° C. for 2 hours. The DNA was then precipitated with ethanol and pelleted. The DNA pellet was resuspended in 27 μl of water and quantified using a NanoDrop 2000 spectrophotometer. Verification of DNA was done by adding 2U DNase (Sigma D5307-1000UN) to digest the resuspended pellet for 2 hours at 25° C., followed by a second round of ethanol precipitation.

Glucose oxidase activity was measured using a colorimetric assay. Reactions containing 0.2% glucose, 75 mM sodium acetate buffer (pH 5.2), CHAPSO and 6NβCD at the indicated concentrations, and glucose oxidase (Sigma, cat no. G7141), were incubated at 42° C. for 1 hour in a total volume of 10 μl. Five microliters of each reaction were added to a clear 96-well plate containing 100 μl of 50 mM HCl. Then, 10 μl each of 1 mM ammonium heptamolybdate, 5% starch solution (Sigma, cat no. S9765), and 1 M sodium iodide were added to each well. The plate was incubated at room temperature for 20 minutes and the absorbance at 570 nm was measured using a BioTek uQuant spectrophotometer.

T4 DNA ligase activity was measured using Lambda phage DNA digested with HindIII as a substrate. Reactions containing 1× T4 ligase buffer (New England Biolabs, cat no. B0202S), 0.5μg digested lambda DNA (New England Biolabs, cat no. N3012L), CHAPSO and 6NβCD at the indicated concentrations, and T4 ligase (New England Biolabs, cat no. M0202L) were incubated at 22° C. for 1 hour in a total volume of 10 μl. Reactions were then heated to 65° C. for 10 minutes to inactivate the T4 ligase. Two microliters of 6× loading dye (Takara Bio, cat no. SD0503) were added to each reaction and 5 μl was loaded onto a 0.5% agarose gel. The gel was run at 3.5 V/cm for 1.5-2 hours and stained with GelRed (Biotium, cat no. 41003). Bands were visualized using a BioRad Gel Doc EZ imager.

### Augmented Immunofluorescence

1 mm³ of formalin-fixed mouse or human brain tissues were dissected and washed in PBS with 0.2% w/v Triton X-100 overnight at 37° C., followed by equilibration in Tris-CAPS buffer (240 mM Tris, 360 mM CAPS, pH 8) and subsequently Tris-CAPS buffer (240 mM Tris, 360 mM CAPS, pH 8) containing 6 mM CHAPSO and 6 mM 6NβCD for 1 hour each at room temperature. The tissue was then transferred to fresh Tris-CAPS buffer with CHAPSO-6NβCD and antibodies at 1:100 dilution along with their corresponding fluorescently-conjugated Fab fragments (Jackson ImmunoResearch) were added. The staining proceeded overnight at RT and the tissue was washed thrice with Tris-CAPS buffer for 1 hour, cleared in OPTIClear, and imaged using a Leica SP8 confocal microscope.

For comparison with conventional immunostaining in PBST, the above procedure was repeated for tissues dissected from adjacent regions of tissues utilized for augmented immunofluorescence, to ensure tissue antigens were present for comparison. The above procedure was replicated for these control samples, except all Tris-CAPS buffer steps (with or without CHAPSO-6NβCD) were replaced with PBS with 0.2% Triton X-100.

### In Situ Recovery of Denatured Antigens Immunofluorescence and Endogenous Fluorescent Protein Signals

1 mm³ of formalin-fixed mouse tissues were obtained. For EYFP, EGFP and mCherry-expressing tissues, Thy1-EYFP brain tissues, Thy1-EGFP brain tissues and CSPG4-dsRed kidney tissues were used. The tissues were first denatured in PBS with 1% w/v Triton X-100 and 6M guanidinium chloride at 60° C. for 1 hour, followed by washing in Tris-CAPS buffer or Tris-CAPS buffer with 6 mM CHAPSO and 6 mM 6NβCD for 2 hours. The incubation solution was then refreshed and 1:100 antibodies along with their corresponding fluorescently-conjugated Fab fragments were added. The staining proceeded overnight at RT, and the tissue was washed thrice with Tris-CAPS buffer for 1 hour, cleared in OPTIClear, and imaged using a Leica SP8 confocal microscope.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### EMBODIMENTS

Embodiment 1. A supramolecular complex augmentor, comprising at least one solubilizing bile salt detergent weakly complexed to at least one molecular host, whereby addition to a solution comprising at least one protein enhances the in vitro performance of the protein.

Embodiment 2. The supramolecular complex augmentor according to embodiment 1, wherein the solubilizing bile salt detergent and the molecular host are combined at a molar ratio of 1:2 to 2:1.

Embodiment 3. The supramolecular complex augmentor according to embodiment 1, wherein the solubilizing bile salt detergent comprises one or more derivatives of cholic acid; C3, C7, or C12epimers of cholic acid; C3, C7, or C12 deoxy derivatives of cholic acid; taurine or glycine conjugates of any derivative; or any combination thereof; the supramolecular complex augmentor having the structure:

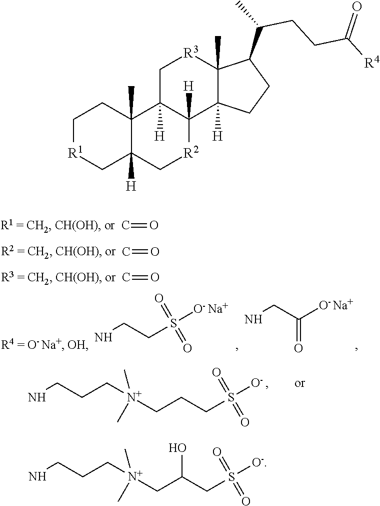

Embodiment 4. The supramolecular complex augmentor according to embodiment 1, wherein the bile salt detergent comprises 3-([3-cholamidopropyl]dimethylammonio)-2-hydroxy-1-propanesulfonate (CHAPSO), 3-([3-cholamidopropyl]dimethylammonio)-1-propanesulfonate (CHAPS), and/or (2-((R)-4-((3R,5S,7R,8R,9S,10S,12S,13R,14S,17R)-3,7,12-trihydroxy-10,13- dimethylhexadecahydro-1H-cyclopenta[a]phenanthren-17-yl)pentanamido)ethyl-(2-(trimethylammonio)ethyl)phosphate) (CHAMP).

Embodiment 5. The supramolecular complex augmentor according to embodiment 1, wherein the molecular host comprises at least one cucurbitril, cyclodextrin, calixarene, cyclophane, cryptand, cryptophane, any derivative thereof, or any combination thereof.

Embodiment 6. The supramolecular complex augmentor according to embodiment 5, wherein the at least one cyclodextrin and/or derivative thereof is of hexakis-β-glucopyranose (α-cyclodextrin), heptakis-β-glucopyranose (β-cyclodextrin), octakis-β-glucopyranose (γ-cyclodextrin), of the structure:

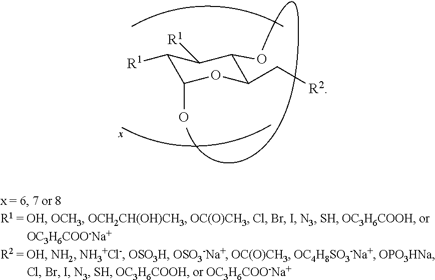

Embodiment 7. The supramolecular complex augmentor according to embodiment 5, wherein the derivative of cyclodextrin is selected from mono-, bis-, tris-, tetrakis-, pentakis-, hexakis-, heptakis- and octakis-substitution of the hydroxyl groups at C2, C3, C6 positions where the groups substituted for the cyclodextrin hydroxy-groups are independently selected from amino-, methoxy-. (2-hydroxy)propoxyl, sulphato-, guanidino-, acetato-, acetamido-, azido-, bromo-, iodo-, chloro-, toluenesulfonyl-, thio-, succinyl-, phosphato-, 4-sulfatobutoxy-, carboxymethoxy-, and (2-aminoethyl)amino-groups.

Embodiment 8. An in vitro property enhanced protein composition, comprising at least one protein and the supramolecular complex augmentor according to embodiment 1, whereby the at least one protein displays enhanced performance in enzyme catalytic activity, binding events, binding affinities, and/or fluorescence over that for optimized conditions absent the supramolecular complex augmentor.

Embodiment 9. A method of enhancing the performance of a protein composition, comprising: providing at least one protein in an environment; and
adding an augmentor according to embodiment 1 to the environment, whereby the enzyme catalytic activity, binding events, binding affinities, and/or fluorescence of the at least one protein is enhanced.

We claim:

1. A supramolecular complex augmentor, comprising at least one solubilizing bile salt detergent complexed to at least one molecular host, whereby addition to a solution comprising at least one protein enhances the in vitro performance of the protein.

2. The supramolecular complex augmentor according to claim 1, wherein the solubilizing bile salt detergent and the molecular host are combined at a molar ratio of 1:2 to 2:1.

3. The supramolecular complex augmentor according to claim 1, wherein the solubilizing bile salt detergent comprises one or more derivatives of cholic acid; C3, C7, or C12 epimers of cholic acid; C3, C7, or C12 deoxy derivatives of cholic acid; taurine or glycine conjugates of any derivative; or any combination thereof; the supramolecular complex augmentor having the structure:

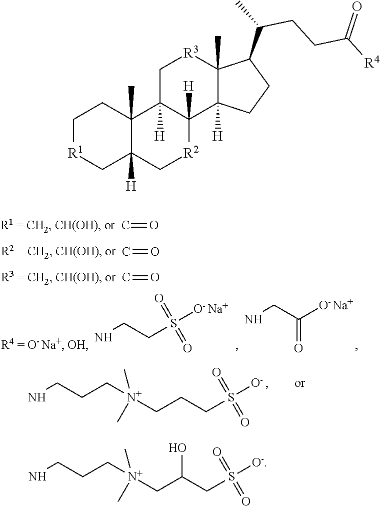

4. The supramolecular complex augmentor according to claim 1, wherein the bile salt detergent comprises 3-([3-cholamidopropyl]dimethylammonio)-2-hydroxy-1-propanesulfonate (CHAPSO), 3-([3-cholamidopropyl]dimethylammonio)-1-propanesulfonate (CHAPS), and/or (2-((R)-4-((3R,5S,7R,8R,9S,10S,12S,13R,14S,17R)-3,7,12-trihydroxy-10,13-dimethylhexadecahydro-1H-cyclopenta[a]phenanthren-17-yl)pentanamido)ethyl-(2-(trimethylammonio)ethyl) phosphate) (CHAMP).

5. The supramolecular complex augmentor according to claim 1, wherein the molecular host comprises at least one cucurbitril, cyclodextrin, calixarene, cyclophane, cryptand, cryptophane, any derivative thereof, or any combination thereof.

6. The supramolecular complex augmentor according to claim 5, wherein the at least one cyclodextrin and/or derivative thereof is of hexakis-β-glucopyranose (α-cyclodextrin), heptakis-β-glucopyranose (β-cyclodextrin), octakis-β-glucopyranose (γ-cyclodextrin), of the structure:

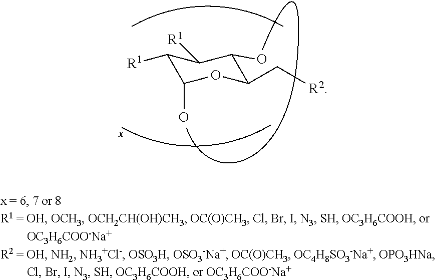

7. The supramolecular complex augmentor according to claim 5, wherein the derivative of cyclodextrin is selected from mono-, bis-, tris-, tetrakis-, pentakis-, hexakis-, heptakis- and octakis-substitution of the hydroxyl groups at C2, C3, C6 positions where the groups substituted for the cyclodextrin hydroxy-groups are independently selected from amino-, methoxy-. (2-hydroxy)propoxyl, sulphato-, guanidino-, acetato-, acetamido-, azido-, bromo-, iodo-, chloro-, toluenesulfonyl-, thio-, succinyl-, phosphato-, 4-sulfatobutoxy-, carboxymethoxy-, and (2-aminoethyl)amino-groups.

8. An in vitro property enhanced protein composition, comprising at least one protein and the supramolecular complex augmentor according to claim 1, whereby the at least one protein displays enhanced performance in enzyme catalytic activity, binding events, binding affinity, and/or fluorescence over that for optimized conditions absent the supramolecular complex augmentor.

9. A method of enhancing the performance of a protein composition, comprising:
providing at least one protein in an environment; and
adding an augmentor according to claim 1 to the environment, whereby the enzyme catalytic activity, binding events, binding affinity, and/or fluorescence of the at least one protein is enhanced.
